(19) United States
(12) Patent Application Publication
(10) Pub. No.: US 2026/0363826 A1
(43) Pub. Date: Nov. 19, 2026

(54) Improvements to Apparatus for use with Teeth

(71) Applicant: Space DP Limited, Wakefield Yorkshire (GB)

(72) Inventors: Samuel Dyson, Wakefield Yorkshire (GB); Affan Saghir, Wakefield Yorkshire (GB); Aaron Holt, Wakefield Yorkshire (GB)

(21) Appl. No.: 19/148,208

(22) PCT Filed: Jan. 17, 2024
(86) PCT No.: PCT/GB2024/050123

§ 371 Date: Jul. 15, 2025

(30) Foreign Application Priority Data
Jan. 17, 2023 (GB) 2300674.5

(51) Int. Cl. A61C 7/08; A61C 19/06

(52) U.S. Cl. CPC A61C 7/08; A61C 19/063

(57) ABSTRACT

The invention relates to apparatus to be worn in the mouth of a user such as an aligner and/or retainer, mouth guard or gumshield for at least some of the user's teeth. The apparatus includes faces which are located so as to be positioned adjacent to the surface of the teeth so as to provide the retaining, aligning and/or protective effect. The apparatus further includes a material which is provided in and/or retained with the apparatus and the material is released from the apparatus and into the mouth of the user when worn so as to reduce the growth of bacteria and/or allow medicine or other treatments to be provided to the user, typically over a period of time of wearing of the apparatus.

The invention to which this application relates is to improvements to apparatus for use with teeth such as for example apparatus commonly known as a teeth aligner or a teeth retainer. A teeth aligner is typically used to allow a relatively discreet progressive adjustment of the orientation of the wearer's teeth over a period of time during which the aligner is worn continuously or at, typically predetermined, intervals. A teeth retainer typically is provided to ensure that the teeth are retained in their current relative positions and without any particular aim of changing the relative positions of the teeth. Another form of the apparatus to which the application relates is worn mainly for protective reasons and is known as a gumshield or mouthguard.

The invention as herein describes is relevant to and is usable in relation to these forms of apparatus which are now referred in a non-limiting manner as apparatus for use with teeth.

The use of aligner apparatus for use with teeth to allow the movement and hence realignment of a number of teeth is well known and typically is recommended by dentists, and mostly in relation to children, so as to allow one or more teeth in a group of teeth, in the bottom and/or top set, to be realigned to improve cleaning and/or an improved visual impression and/or to prevent dental problems from occurring. The use of retainer apparatus for use with teeth can be recommended to be worn to prevent movement of teeth in a group and, in many cases, the apparatus can be recommended to be worn following the realignment which has been achieved by wearing an aligner and so to prevent the teeth returning to their original relative positions.

Gumshields and mouthguards or other forms of apparatus may be required to be worn due to regulations in a sport which is being played by the user, may be recommended for use by a sporting activity and/or may be worn to prevent damage to the teeth such as for example, which may be caused by grinding of teeth.

Typically the apparatus can be provided as two sets, a first set to be worn on all or some of the upper teeth and another set provided to be worn on all or some of the lower teeth. In some cases only one of the sets is required to be used.

The apparatus of this type is now most typically formed of a plastics, polymer material and into which a particular teeth impression suitable for the wearer is made and other formations may also be provided for achieving the location of the wearing of the apparatus is provided. In certain instances, a series of the apparatus can be provided as part of a kit and the apparatus is worn in sequence so as to, for example, gradually align the teeth to the required setting. Typically the apparatus is transparent, which has an improved visual appearance with respect to conventional frames formed of metal and, as a result of this, it is found that the apparatus is more likely to be worn by the user and, as a result is more likely to be effective in allowing the desired aim with respect to the group of teeth to be achieved.

A problem with the use of this form of apparatus is that the same need to be closely fitted on the wearer's set of teeth, and, as a result, the inner walls of the apparatus, which are those walls which contact against the outer surface of the front and rear faces of the teeth, often do not contact uniformly across all of the surfaces of the teeth and so it is found that, in use, small and relatively difficult to access gaps are formed between the surface of the teeth and the inner wall of the aligner or retainer. These gaps allow the harbouring of bacteria and encourage bacteria growth which can be harmful to the teeth and such as, for example, the enamel surfaces of the teeth. This problem is especially prevalent if the apparatus is worn for a longer period of time than advised and/or if the apparatus and/or teeth are not cleaned properly or as frequently as advised. Thus, the benefits of the realignment or retention of the teeth position which can be achieved, may be accompanied by disadvantages of damage which may be caused to the teeth.

An aim of the present invention is therefore to provide the apparatus which allows the proposed benefits of wearing the same, such as alignment, retention and/or protection to be achieved, whilst, at the same time, minimising the potentially damaging effect of the growth and retention of bacteria and/or harmful substances being located by the apparatus in proximity to the wearer's teeth.

A further aim of the present invention is to provide a means to distribute medication and/or other treatments orally to a user when wearing, via their wearing of the apparatus, and thereby avoid the need for the user to separately take the medicine or treatment through swallowing tablets or the like. A further aim is to allow the medicine or treatment to be provided ion a controlled and/or slow release manner over a period of time to thereby improve the effectiveness of the medication or treatment.

In a first aspect of the invention, there is provided apparatus to be worn on at least a group of a user's teeth and in the mouth of said user, said apparatus including at least one face which is located so that at least one face of the apparatus is positioned adjacent to external surfaces of at least some of the user's teeth in use and wherein the apparatus includes a material located therewith to be distributed from the said apparatus within the mouth of the user when the apparatus is located therein.

In one embodiment the said material is located on at least one portion of a face of the apparatus and/or is embedded within the apparatus so as to be integral therewith.

In one embodiment the apparatus includes one or more retaining means formed at one or a series of locations and which are provided for the receipt of a portion of the said material and to assist in the retention of the material in position during the wearing of the apparatus.

In one embodiment the said one or more retaining means are selectively variable in size so as to locate portions of the said material of variable sizes in use.

In one embodiment the retaining means includes a seat or pocket for the material. In another embodiment, the retaining means and/or material includes an adhesive such that the material may be adhered in location on the face of the apparatus.

Typically when the material is to be provided at locations on the apparatus face or faces rather than as a uniform coating the particular locations may be selected to suit particular uses and/or ensure that the said material is provided to act at one or more specified locations where bacterial growth is known to be prevalent. Typically the parameters which are referred to when deciding upon the location include the location of the material on the apparatus, the quantity of the material added at each location, the area of the said location for the shape of the portion of the material and/or the number of portions of the said material. Typically, the said face of the apparatus including the one or more retaining means faces towards the teeth of the user when the apparatus is worn.

In one embodiment, the said material portion is provided in a form of a cake or tablet which, when located with the apparatus and worn in the mouth, dissolves upon contact with saliva over time when worn.

Typically the cake or tablet format of the material is produced in the form of a paste and then formed & dried under pressure to create the tablet format of the material.

In one embodiment the said material is coated on the apparatus so as to be located across the surface of the same.

In one embodiment the said material is a gel or other fluid material which has sufficient viscosity so as to be retained on the face of the apparatus during fitting and wearing of the same.

In another embodiment the material is provided as a spray. Typically the spray is produced using an alcohol or water base which, when the material is sprayed onto the apparatus, evaporates and so leaves a relatively uniform coating of the active or key components on the surface of the apparatus. In one embodiment the spray may be applied to the apparatus under factory conditions prior to the finishing & packaging of the apparatus for subsequent use.

In one embodiment the said material is mixed with the components which are used to form the apparatus and typically when the components are in a liquid form, so that said material is provided as an integral part of the apparatus.

In a further embodiment the said material is impregnated into the apparatus such that the said material is slowly released from the apparatus faces during use. In this way, the apparatus acts as a slow-release mechanism instead of providing the material as a separate part applied to and/or located with the apparatus.

In one embodiment the material is provided as a stratified layer of the apparatus.

In one embodiment the material is configured to be released within the mouth of the user over a period of time. Preferably said period of time or a combination of periods of time is equivalent to or exceeds the recommended duration of wearing of the apparatus so as to be released over that time.

Typically the duration of the beneficial effect provided by the said material is such as to allow the recommended duration of wearing the apparatus between cleans to be increased in comparison to conventional advice for the wearing of the apparatus.

Typically the material can be provided in a range of formats such as a gel, dry coating tablet or cake and/or a spray and provided for use in conjunction with the apparatus.

Typically said material which is provided in conjunction with the apparatus includes one or more components which allow the material to be in the required format for subsequent use. For example, the gel format can be glycol or water based and preferably the gel is of a relatively high viscosity in order to be able to maintain the adhesion between the gel and the apparatus and tooth and therefore prolong the effectiveness of the gel material.

In one embodiment, the material is or includes an anti-microbial material so as to deter the harbouring and/or growth of potentially harmful bacteria and/or other micro-organism substances in the mouth in use.

In one embodiment, the potentially harmful effect which is prevented is damage to the enamel of the external faces of the teeth being caused by the bacteria harboured between the teeth and the said apparatus.

Typically, the material includes any or any combination of the components Sodium Bicarbonate, Calcium Hydroxyapatite and Sodium Fluoride. Preferably said material includes 500 ppm of the said components.

In another embodiment, the material is or includes medication to be administered orally over the duration of wearing the apparatus.

In another embodiment, the material is or includes an aroma-infused polymer. Typically the polymer is infused with natural, non-toxic aromas such as mint, citrus or the like.

In another embodiment, the material is or includes pH balancing agents. Typically the pH balancing agent includes fluoride.

In one embodiment, the apparatus is formed of a polymer including an anti-bacterial compound. Typically, the anti-bacterial compound includes silver ion compounds.

In one embodiment, the material provided in and/or on the apparatus, is of a form so as to provide a “slow release” of the material into the mouth and/or onto the teeth of wearer whilst the apparatus is being worn.

In one embodiment the apparatus includes a first set of apparatus including a first wall having first and second opposing faces and a second wall having third and fourth opposing faces, said first and second walls spaced apart, typically by a base, so as to define a gap between the second and third faces and in which gap the user's teeth are located when the apparatus is worn in the mouth of the user. In one embodiment the said first and fourth faces of the apparatus face away from the teeth and into the mouth of the user when the apparatus is worn.

In one embodiment the apparatus includes a second set with first, second, third and fourth walls.

Typically the apparatus can be selectively worn in a configuration with only the first set or in a configuration in which the first and second sets are worn on the upper teeth and lower teeth respectively.

Typically the said material is provided on the apparatus so as to be exposed at at least one of the said first, second, third or fourth faces of the first and/or second sets of the apparatus, or any combination thereof.

In one embodiment the apparatus is at least partially smoothed by a vapour smoothing process. Typically, the finishing of at least those faces to be adjacent to the surfaces of the teeth includes the performance of vapour smoothing of surfaces of the apparatus which, when worn, are exposed and/or contact with the teeth.

In one embodiment the vapour smoothing process utilises acetone or another similar reactive vapor which is applied to said surfaces and which smooths the surface of the apparatus, so as, for example, to remove 3D print lines and/or relatively rough surfaces so as to reduce the area size and locations at which bacterial growth is most likely.

In one embodiment, the apparatus includes one or more pressure distribution pads and/or areas of increased thickness. Typically the pressure distribution pads are selectively movable with respect to the apparatus and/or adjustable in thickness. In one embodiment, the apparatus includes at least one channel therethrough for the passage of liquid towards and/or away from the teeth.

In one embodiment, the apparatus includes at least one indicator located therewith. Typically the indicator is configured to visually vary depending on the pH level and/or moisture level of the mouth of the user in use.

In one embodiment, the apparatus includes a ridge member that is adjacent to the tongue of the user in use. Typically, the ridge is configured to direct the tongue of the user away from any edges of the apparatus or other areas which may be sharp or rough and cause potential abrasion when the apparatus is being worn.

In one embodiment, the apparatus includes at least one removable insert member. Typically the at least one insert member includes the said material to be released into the users mouth in use.

In one embodiment, the apparatus takes the form of any of an aligner, a retainer, a mouth guard, or a gum shield.

In one embodiment, the apparatus includes at least one locating means to engage with the gums and/or teeth of the user to substantially locate the apparatus with the said teeth and mouth of the user in use.

Typically the locating means includes a form fitting portion configured to fit around at least one tooth and/or the gum of the user to selectively locate the apparatus with the same. Preferably the form fitting portion creates an at least partial vacuum seal with the at least one tooth and/or the gum of the user with which it is located in use.

In another embodiment, the locating means includes a friction fit and/or adhesive portion to selectively locate the apparatus with the gums and/or teeth of the user.

Specific embodiments of the invention are now described with reference to the accompanying drawings; wherein

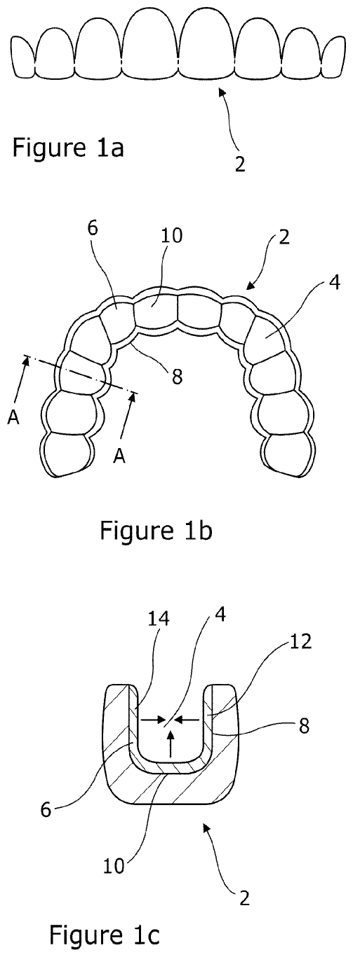

FIGS. 1a-c illustrate views of apparatus and including material to be distributed within the mouth of a user in accordance with one embodiment of the invention;

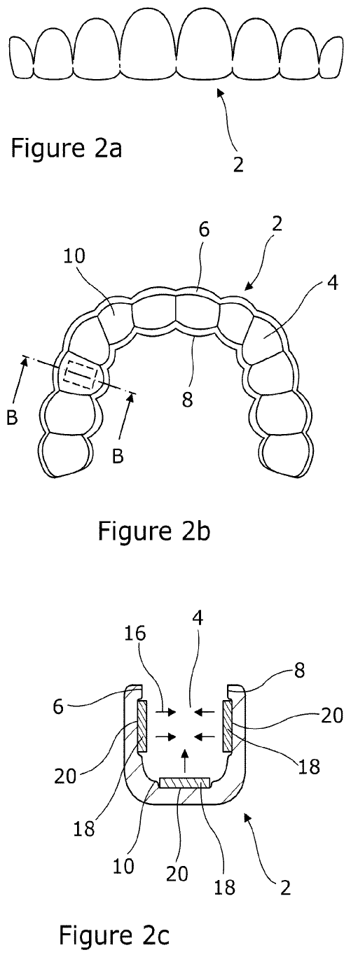

FIGS. 2a-c illustrate views of apparatus and including material to be distributed within the mouth of a user in accordance with a further embodiment of the invention;

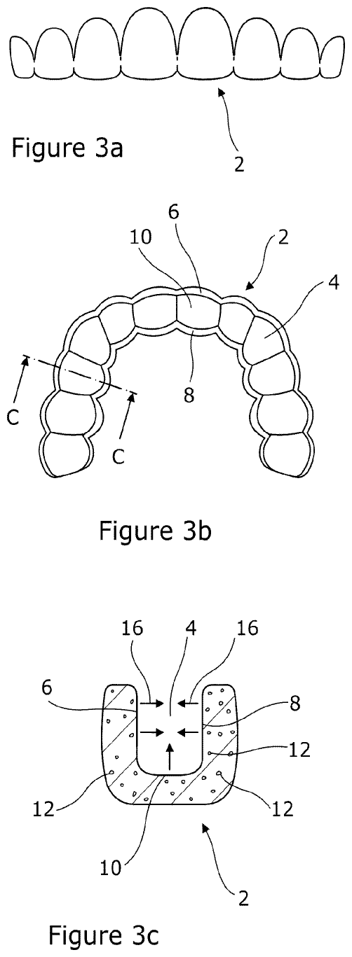

FIGS. 3a-c illustrate views of apparatus and including material to be distributed within the mouth of a user in accordance with a yet further embodiment of the invention; and

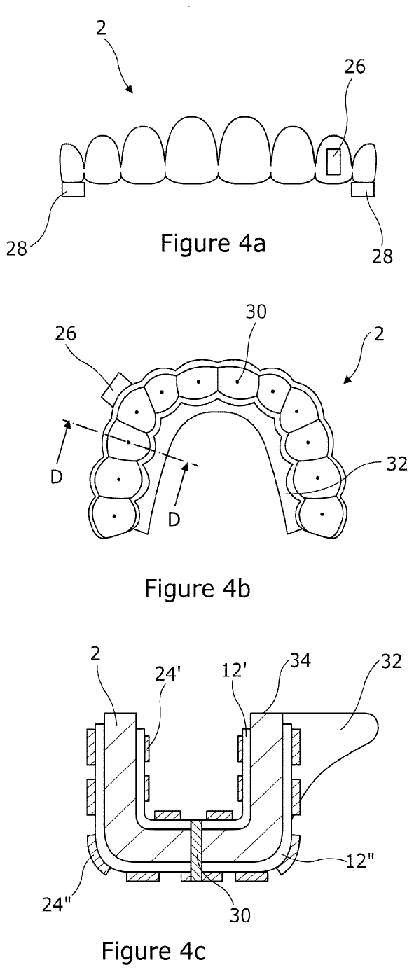

FIGS. 4a-c illustrate views of apparatus and including material to be distributed within the mouth of a user in accordance with a yet further embodiment of the invention.

In each of the embodiments shown in the Figures there is shown a front elevation, plan and cross sectional view of a first set of the apparatus to be worn on teeth, in the form of an aligner. In alternative embodiments, the apparatus may be a retainer, gum shield and/or mouth guard and the invention as herein described relates equally to and may be implemented in, these other forms of the apparatus It should also be appreciated that the said first set which is shown may be selectively used on its own or in conjunction with a second set with similar features to those as herein described for the first set, with the first set being worn on the lower or upper teeth and the second set, when selected, being worn on the other of the lower or upper teeth.

The apparatus comprises a body 2 formed of a polymer material and which forms a recessed channel 4 in and along which the teeth of the wearer are received when the apparatus is worn. The particular shape of the recess is moulded so as to replicate the shape of the wearer's teeth so as to ensure a close fit between the second and third inner wall faces 6,8 and base 10 of the apparatus with the external surfaces of the teeth. This close fit ensures that there is at least a partial vacuum formed between the aligner and the teeth, ensure that the aligner is selectively retained with the same. In an alternative embodiment, the aligner may include friction and/or adhesive portions to locate the same with the teeth and/or gums of the user. The body 2 is relatively rigid so as to retain the apparatus in location and also be able to exert an alignment or retention force to the teeth when worn.

Despite the close fit there are inevitably gaps between the teeth and the body and/or rough surfaces which are known to harbour and encourage growth of bacteria.

In accordance with the invention and each of the embodiments shown, the apparatus includes a material provided to inhibit or prevent the bacterial growth. However, it will also be appreciated that the material to be distributed may also be any of medicine to be administered orally to the user, an aroma-infused substance such as a mint, or the like. The skilled user will also appreciate that the material to be distributed may be any combination of these.

In FIGS. 1a-c there is shown a first embodiment in which the said material 12 is provided so to form an external coating 14 on the faces 6,8 and base 10 so that the material passes as indicated by arrows 16, as shown in the cross sectional view along line AA, and into the gaps between the inner faces and base 6,8,10 and the surfaces of the user's teeth and onto the said teeth surfaces so as to reduce or prevent bacterial growth. The material 12 may be applied across the face and/or base as a gel or as a spray which is applied to the faces 6, 8, and/or base 10 prior to the wearing of the body 2 on the teeth.

In FIGS. 2a-c there is shown a second embodiment in which the said material 12 is provided as a series of tablets or cakes 18 which are held in respective location means 20. Typically the location means are provided at spaced intervals around the recess 4 so that the material passes as indicated by the arrows 16 along the length of the recess and into the gaps between the inner faces 6,8 and base 10 and the surfaces of the users teeth and hence reduces or prevents bacterial growth. The location means 20 may be seats for the tablets or cakes 18, or portions of an adhesive material to adhere the tablets or cakes 18 to the retainer 2. The seats or pockets 20 may be formed of a malleable material such as a polymer, such that the size of the same may be adjusted by a user to adapt the pocket or seat 20 for the reception of a larger tablet 18 therewith.

In FIGS. 3a-c there is shown a third embodiment in which the said material 12 is provided as a series of particles which can be ionically impregnated into the material used to form the body 2. This allows the material 12 to pass as indicated by the arrows 16 along the length of the recess and into the gaps between the faces 6,8 and base 10 and the surfaces of the users teeth and hence reduces or prevents bacterial growth.

In FIGS. 4a-c there is shown a fourth embodiment of the invention in which said material 12 is provided as a first layer 12′ on the faces 6,8 and/or base 10 and as a second layer 12″ on the first and/or fourth outer face 22 of the apparatus 2. The first layer 12′ and second layer 12″ are then coated in a respective mesh layer 24′,24″ such that the material 12 is applied in a stratified manner to the apparatus 2. This stratified construction slows the rate at which the material 12 is distributed within the mouth of the user as the mesh layer reduces the transfer rate through the same.

In this embodiment, the retainer 2 includes an indicator 26 such as a strip containing phenolphthalein that is adhered to the outer face 22 of the apparatus 2. The phenolphthalein strip will visually indicate to the user when the pH within the mouth is too acidic, so that pre-emptive steps may be made such as, for example, brushing the teeth with an alkaline toothpaste.

The aligner 2 in this embodiment further includes pressure distribution pads 28 located on the underside of the same. When the user bites down on these pads with their molar teeth, the apparatus 2 flexes, relieving pressure on the front teeth of the user. The pressure pads 28 are adhered to the underside of the aligner using an adhesive, and may be relocated to align with different teeth of the user. The pads may be swapped for other pads of varying thicknesses to vary the flexing of the aligner and therefore the pressure relief experienced by the user.

The apparatus 2 in this embodiment further includes a plurality of hydration channels 30. These channels 30 extend through the depth of the apparatus 2, typically between the inner and outer faces of a wall, and allow moisture to pass from one side of the apparatus to the other. This allows for saliva circulation within the mouth of the user, helping to maintain the natural oral hydration and minimise dryness within the mouth.

The apparatus 2 further includes a ridge member 32 that extends inwardly from the upper edge of the apparatus 2. This ridge member 32 acts to guide the tongue of the user away from the sharp edge running along the upper edge of the aligner 2. The ridge member 32 may be formed of a relatively soft material with an ergonomic profile, providing comfort to the user against the faces of the apparatus.

In another example, the apparatus 2 may be provided as a kit including a cleaning insert. This insert may be provided to be placed within the apparatus when the apparatus is removed from the mouth of the user, and contains anti-microbial material. As such, the insert acts to ensure the apparatus 2 remains suitably free from bacteria when being worn but preferably also between uses of the apparatus.

1. Apparatus to be worn on at least a group of a user's teeth and in the mouth of said user, said apparatus comprising:
at least one face located so that at least one face of the apparatus is positioned adjacent to external surfaces of at least some of the user's teeth in use and wherein the apparatus includes a material located therewith to be distributed from the apparatus within the mouth of the user when the apparatus is located therein.

2. Apparatus according to claim 1, wherein the material is retained on at least one portion of at least one face of the apparatus and/or is embedded within the apparatus.

3. Apparatus according to claim 1, wherein the apparatus includes retaining means formed at one or a series of locations on the apparatus and which receive a portion of the material and retain at least part of the material in position for a period of time during wearing of the apparatus in the user's mouth.

4. Apparatus according to claim 3, wherein the one or more retaining means are selectively variable in size.

5. Apparatus according to claim 3, wherein the retaining means is or includes an adhesive location such that the material is adhered in position with respect to the retaining means.

6. Apparatus according to claim 1, wherein the material is coated on at least one face or portion of the face of the apparatus.

7. Apparatus according to claim 1, wherein the material is incorporated with materials from which the apparatus is formed so that the material is provided as an integral part of the apparatus.

8. Apparatus according to claim 7, wherein the material is provided as a stratified layer of the apparatus.

9. Apparatus according to claim 1, wherein the said material is progressively released from the apparatus over a period of time of use of the apparatus is the user's mouth.

10. Apparatus according to claim 1, wherein the apparatus includes a first set of apparatus including a first wall having first and second opposing faces and a second wall having third and fourth opposing faces, said first and second walls spaced apart so as to define a gap between the external surfaces of the second and third faces in which the user's teeth are located when the apparatus is worn in the mouth of the user.

11. Apparatus according to claim 10, wherein the first and fourth faces of the apparatus face into the mouth of the user when the apparatus is worn.

12. Apparatus according to claim 10, wherein the apparatus includes a second set of apparatus with first, second, third and fourth walls.

13. Apparatus according to claim 12 wherein the apparatus is selectively worn in a configuration with only the first set or in a configuration in which the first and second sets are worn.

14. Apparatus according to claim 10 wherein the material is provided on the apparatus so as to be exposed at at least one of the first, second, third or fourth faces of the first and/or a second set of the apparatus, or any combination thereof.

15. Apparatus according to claim 1, wherein the material is provided in any format of a gel, dry coating tablet or cake and/or a spray.

16. Apparatus according to claim 1, wherein the material includes any or any combination of medication to be administered orally, an aroma-infused polymer and/or a pH balancing agent.

17. Apparatus according to claim 1, wherein the apparatus is formed of a polymer

18. Apparatus according to claim 1, wherein the material includes an anti-microbial material so as, when released from the apparatus into the mouth, to deter the harbouring and/or growth of potentially harmful bacteria and/or other micro-organism substances in the mouth in use.

19. Apparatus according to claim 18, wherein the material includes the components Sodium Bicarbonate, Calcium Hydroxyapatite or Sodium Fluoride.

20. Apparatus according to claims 18, wherein the material includes at least one silver ion compound.

21. Apparatus according to claim 1, wherein at least one of the apparatus faces is at least partially smoothed by a vapour smoothing process.

22. Apparatus according to claim 1, wherein the apparatus includes one or more pressure distribution pads.

23. Apparatus according to claim 1, wherein the apparatus includes at least one channel to allow the passage of liquid towards and/or away from the teeth of the user when the apparatus is being worn.

24. Apparatus according to claim 1, wherein the apparatus includes at least one pH indicator and/or at least one moisture indicator located thereon.

25. Apparatus according to claim 1, wherein the apparatus includes a ridge member on a surface of the apparatus that is adjacent the tongue of the user in use.

26. Apparatus according to claim 1, wherein the apparatus includes at least one removable insert member including the material to be released into the user's mouth in use.

27. Apparatus according to claim 1, wherein the apparatus takes a form of any of an aligner, a retainer, a mouth guard, or a gum shield.

28. Apparatus according to claim 1, wherein the apparatus includes at least one locating means to engage with gums and/or teeth of the user to substantially locate the apparatus within the mouth of the user in use.
